(19) United States
(12) Patent Application Publication
(10) Pub. No.: US 2020/0199646 A1
(43) Pub. Date: Jun. 25, 2020

(54) PORTABLE UREA SENSOR USING UREASE-IMMOBILIZED INSOLUBLE POROUS SUPPORT

(71) Applicant: INDUSTRY ACADEMIC COOPERATION FOUNDATION, HALLYM UNIVERSITY, Chungcheon-si ,Gangwon-do (KR)

(72) Inventors: Gun Yong SUNG, Seoul (KR); Chan Hum PARK, Chuncheon-si (KR); Jung Eun LEE, Guri-si (KR); Bo Mi MOON, Seoul (KR)

(21) Appl. No.: 16/071,065

(22) PCT Filed: Aug. 8, 2016
(86) PCT No.: PCT/KR2016/008692

§ 371 Date: Jul. 18, 2018

(30) Foreign Application Priority Data
Jan. 18, 2016 (KR) 10-2016-0005717

(51) Int. Cl. C12Q 1/34; G01N 33/62; B01L 3/00; G01N 27/07; G01N 33/493

(52) U.S. Cl. CPC C12Q 1/34; G01N 33/62; B01L 3/502715; B01L 2300/0645; G01N 33/493; B01L 2300/12; G01N 27/07

(57) ABSTRACT

The present invention relates to a small urea sensor module, configured such that a urease-immobilized insoluble porous support, in which urease is immobilized on a porous support made of a natural polymer such as silk fibroin, etc. or a synthetic polymer, is mounted in a fluidic chamber and also such that the electrode surface of a three-electrode strip is exposed to the bottom surface of the chamber. This urea sensor is essential for the evaluation of the regenerated solution from a portable peritoneal dialysis fluid regeneration system and has advantages such as portability, reproducibility, mass productivity and simplicity, which will greatly contribute to disease management of patients with chronic renal disease.

### TECHNICAL FIELD

The present invention relates to a portable urea sensor module using a urease-immobilized insoluble porous support.

### BACKGROUND ART

The kidneys basically function to discharge waste and also regulate physiological functions essential for the health of various organisms. The kidneys in the human body are responsible for the discharge of nitrogen waste and certain organic compounds, maintenance of volume homeostasis, regulation of osmotic pressure, acidity, divalent cations, phosphorus, potassium and blood pressure, erythropoiesis control, vitamin D synthesis, antibody expression, immune regulation, redox balance adjustment, etc. About 10 functions, including passive filtration and active reabsorption, for over 100 L/day of body fluids are carried out in a small amount of kidney tissue weighing only hundreds of grams.

Since patients with end-stage renal disease need to go to the hospital for hemodialysis for 4 hr three times a week or to replace 2 L of peritoneal dialysis fluid four times a day at home, great limitations are imposed on reducing the cost and inconvenience thereof. Hence, there is an urgent need for a simple, portable, and inexpensive dialysis technique. A portable urea sensor technique, which accurately and easily detects urea concentration in the development of such a portable artificial dialysis system, is one key urea technique.

A recently reported urea sensor is configured such that urease is immobilized on the surface of a support made up of nickel oxide nanoparticles or a nickel oxide thin film having a large surface area so that an oxidation reaction, occurring upon decomposition of urea into ammonia and carbon dioxide, is electrochemically measured through a nickel oxide electrode, whereby the urea concentration is detected based on the measured oxidation current value.

The development of urea sensors is described below.

In 1995, Boubriak et al. of the Institute of Molecular Biology & Genetics of Ukraine manufactured and publicized a biosensor, configured such that urease is immobilized on a bovine serum albumin osmosis membrane attached to an ISFET to detect the urea in the serum.

In 2002, Gambhir et al. of the National Institute of Physics of India manufactured and publicized a urea electrochemical biosensor (detection range: 5×10⁻³ mol/l to 6×10⁻² mol/l), configured such that PPY microparticles covalently bonded to urease are attached to the surface of conductive polypyrrole-polyvinyl sulfonate electrochemically applied on ITO.

In 2011, Gabrovska et al. of Zlatarov University of Bulgaria manufactured and publicized a urea biosensor having a detection limit of 0.5 mM and a sensitivity of 3.1927 μAmM⁻¹cm⁻², configured such that a polymer osmosis membrane is chemically modified to chemically immobilize urease thereon and also such that rhodium nanoparticles, which convert ammonia decomposed by urease into nitrogen, are immobilized on the osmosis membrane.

In 2013, Tak et al. of Delhi University of India manufactured and publicized a urea electrochemical biosensor (43.02 μAmM⁻¹cm⁻²), configured such that ZnO/ITO and ZnO-MWCNT nanocomposite/ITO is coated with urease.

In 2013, Laurinavicius et al. of Vilnius

University of Lithuania publicized a urea electrochemical sensor (linearity up to 5 mM) using a reverse osmosis membrane chemically immobilized with a carbon black paste electrode and urease.

### DISCLOSURE

### Technical Problem

Accordingly, the present invention is intended to provide an economical portable urea sensor module, in which urease is immobilized on an insoluble porous support in a simple manner and the urease-immobilized insoluble porous support is placed in a small fluidic chamber with electrodes so that a urea concentration may be readily detected.

### Technical Solution

The present inventors have manufactured a portable urea sensor module in a manner in which, in lieu of a nickel oxide electrode in order to overcome defects of nickel oxide electrode materials, a porous silk fibroin disk the surface of which is immobilized with urease is mounted in a microfluidic chamber and a screen-printed three-electrode strip is employed, thus detecting urea.

The present inventors have manufactured a portable urea sensor configured such that urease is immobilized on a porous silk fibroin disk in order to apply the same to a wearable artificial kidney system based on peritoneal dialysis. The porous structure of the silk fibroin disk is manufactured using a salt-leaching process. The disk acts as an effective matrix for immobilizing urease (Ur), which is used to detect urea. A PDMS (polydimethylsiloxane) fluidic chamber, provided with three electrodes screen-printed on a single strip and a urease-immobilized porous silk fibroin disk, is employed to detect urea using cyclic voltammetry (C-V). The manufactured urea sensor exhibits high sensitivity and shows a linear dependence of current on the urea concentration.

### ADVANTAGEOUS EFFECTS

According to the present invention, a urea sensor module can exhibit high sensitivity and a linear dependence of current on the urea concentration. Therefore, the urea sensor module of the present invention is essential for the evaluation of the regenerated solution from a portable peritoneal dialysis fluid regeneration system and also has advantages such as portability, reproducibility, mass productivity and simplicity, which will greatly contribute to disease management of patients suffering from chronic renal disease.

### DESCRIPTION OF DRAWINGS

FIG. 1 schematically shows (A) a process of manufacturing a silk fibroin support, and (B) an enzyme immobilization process;

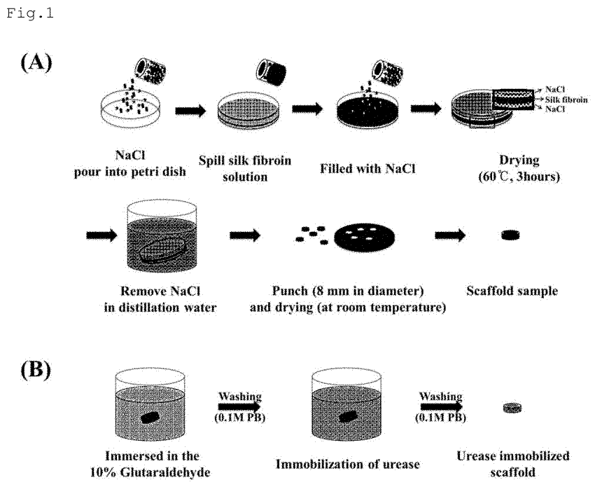

FIG. 2 shows schematic views and a photograph of a urea sensor module according to the present invention, (A) illustrating the overall configuration, (B) illustrating the top plan view, (C) illustrating the side view, and (D) illustrating the actually manufactured module photograph;

FIG. 3a is a graph showing changes in current and voltage depending on the urea concentration; and

FIG. 3b is a graph showing changes in oxidation current with measurement time depending on the urea concentration at 1 V.

### BEST MODE

The present invention provides a portable urea sensor module, comprising:

a fluidic chamber;

an electrode strip fixed at the bottom of the fluidic chamber and configured such that a reference electrode, a cathode and a anode are screen-printed;

a urease-immobilized insoluble porous support positioned in the fluidic chamber;

a sample inflow tube configured to enable a sample to flow into the fluidic chamber;

and a sample outflow tube configured to enable a sample to flow out from the fluidic chamber.

Also, in the portable urea sensor module according to the present invention, the fluidic chamber is made of a synthetic resin material.

Also, in the portable urea sensor module according to the present invention, the fluidic chamber is made of PDMS (polydimethylsiloxane).

Also, in the portable urea sensor module according to the present invention, the urease-immobilized insoluble porous support may be exchanged as necessary. Specifically, the urease-immobilized insoluble porous support is placed in the fluidic chamber upon urea detection testing, and may be exchanged before deterioration of the urea detection function thereof. The urease-immobilized insoluble porous support may be shaped so as to have the same cross-section as the cross-section of the fluidic chamber, whereby it is stably positioned in the fluidic chamber and contains urease in as large an amount as possible. For example, when the fluidic chamber is in a cylindrical shape, the urease-immobilized insoluble porous support is preferably shaped in the form of a disk.

Also, in the portable urea sensor module according to the present invention, the electrode strip, which is fixed at the bottom of the fluidic chamber and is configured such that a reference electrode, a cathode and a anode are screen-printed, extends outside of the fluidic chamber so as to measure an oxidation current value.

The insoluble porous support for immobilizing urease may be made of at least one biocompatible material selected from the group consisting of fucoidan, collagen, alginate, chitosan, hyaluronic acid, silk fibroin, polyimide, polyamic acid, polycaprolactone, polyetherimide, nylon, polyaramid, polyvinyl alcohol, polyvinylpyrrolidone, poly-benzyl-glutamate, polyphenylene terephthalamide, polyaniline, polyacrylonitrile, polyethylene oxide, polystyrene, cellulose, polyacrylate, polymethyl methacrylate, polylactic acid (PLA), polyglycolic acid (PGA), polylactic acid-co-polyglycolic acid (PLGA), poly{poly(ethylene oxide) terephthalate-co-butylene terephthalate} (PEOT/PBT), polyphosphoester (PPE), polyphosphazene (PPA), polyanhydride (PA), poly(ortho ester) (POE), poly(propylene fumarate)-diacrylate (PPF-DA), and poly(ethylene glycol) diacrylate (PEG-DA).

In addition, the present invention provides a urease-immobilized insoluble porous support, configured such that urease is immobilized on an insoluble porous support made of at least one biocompatible material selected from the group consisting of fucoidan, collagen, alginate, chitosan, hyaluronic acid, silk fibroin, polyimide, polyamic acid, polycaprolactone, polyetherimide, nylon, polyaramid, polyvinyl alcohol, polyvinylpyrrolidone, poly-benzyl-glutamate, polyphenylene terephthalamide, polyaniline, polyacrylonitrile, polyethylene oxide, polystyrene, cellulose, polyacrylate, polymethyl methacrylate, polylactic acid (PLA), polyglycolic acid (PGA), polylactic acid-co-polyglycolic acid (PLGA), poly{poly(ethylene oxide) terephthalate-co-butylene terephthalate} (PEOT/PBT), polyphosphoester (PPE), polyphosphazene (PPA), polyanhydride (PA), poly(ortho ester) (POE), poly(propylene fumarate)-diacrylate (PPF-DA), and poly(ethylene glycol) diacrylate (PEG-DA) and provided in the form of a film.

### Mode for Invention

A better understanding of the present invention will be given through the following examples, which are set forth to illustrate but are not to be construed as limiting the scope of the present invention, as will be apparent to those skilled in the art. Particularly in examples of the present invention, silk fibroin is adopted as a material for an insoluble porous support, but the insoluble porous support is not limited only to silk fibroin, and a fluidic chamber has a cylindrical shape, but a polyprism shape may also be applied, in addition to the cylindrical shape, upon real-world application thereof.

### 1. Manufacture of Urease-Immobilized Porous Silk Fibroin Disk

In order to manufacture a three-dimensional porous support, salt was spread in a petri dish and a silk fibroin aqueous solution was then poured thereon, after which the petri dish was filled with salt. The resulting mixture was dried in an oven at 60° C. for 3 hr or more and thus hardened. The salt of the dried mixture was immersed in distilled water and thus removed. While distilled water was frequently exchanged for 36 to 72 hr, salt was removed. The salt-free plate-shaped support was punched with a punch having a diameter of 8 mm and then dried at room temperature or lyophilized, thereby manufacturing a three-dimensional porous silk fibroin support.

The silk fibroin support thus manufactured was immersed in a 10% glutaraldehyde solution and activated with stirring at 30° C. for 1 hr. Thereafter, unreacted glutaraldehyde was washed with a 0.1 M phosphoric acid buffer. The support activated due to glutaraldehyde was transferred into a urease solution and immobilized with stirring at room temperature for 2 hr. In order to remove the urease that was not immobilized on the support, washing with a 0.1 M phosphoric acid buffer was performed several times, followed by drying at 4° C. and storage.

### 2. Manufacture of Portable Urea Sensor and Measurement of Urea Concentration

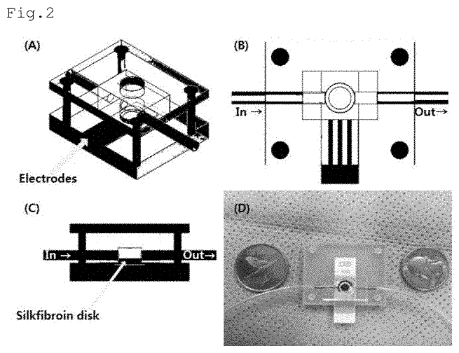

In order to measure the urea concentration in the sample solution, an electrode strip configured such that a reference electrode, a cathode and a anode were screen-printed was fixed at the bottom of a cylindrical microfluidic chamber made of PDMS (diameter: 8 mm, height: 3.5 mm), the silk fibroin disk was placed in the fluidic chamber, and a fluid transfer tube was connected thereto, after which the portable urea sensor module shown in FIG. 2 was manufactured by assembling the module with fixing screws using a test jig made using a 3D printer.

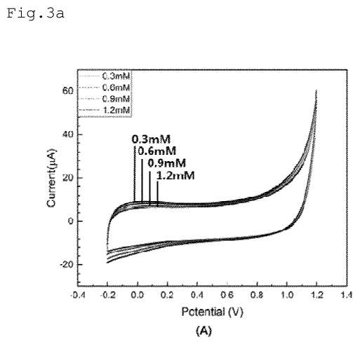

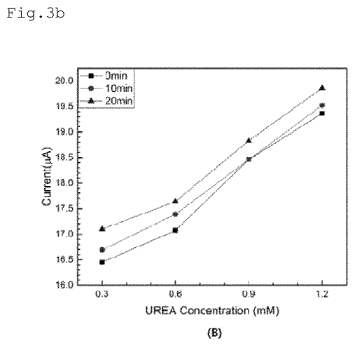

The electrodes of the manufactured sensor module were connected to potentiostats, after which each of the urea samples having different concentrations was introduced through the fluid transfer tube, and a cyclic voltammogram (C-V) thereof was measured three times at a scan rate of 0.05 V/sec in the range of −0.2 V to 1.2 V at intervals of 10 min. FIG. 3a is a graph showing C-V measured depending on the concentration, and FIG. 3b is a graph showing the oxidation current with measurement time depending on the urea concentration at 1 V.

Based on the results of electrochemical measurement using the portable urea sensor module, including the microfluidic chamber provided with the urease-immobilized porous silk fibroin disk and the screen-printed three-electrode strip, the concentration-oxidation current was linearly shown in the urea concentration range of 0.3 to 1.2 mM, and the sensitivity was 23 (μAmM⁻¹cm⁻²) .

### INDUSTRIAL APPLICABILITY

A portable urea sensor module of the present invention is useful to check conditions of renal disease patients.

1. A urea sensor,
a fluidic chamber with an inlet and an outlet;
an electrode strip fixed in the fluidic chamber and comprising a reference electrode, a cathode and an anode;
a urease containing device placed in the fluidic chamber;
a liquid inflow tube connected to the inlet and configured to supply urea-containing liquid into the fluidic chamber; and
a liquid outflow tube connected to the outlet and configured to discharge the urea-containing liquid out of the fluidic chamber,
wherein the urea sensor is configured:
such that the urease-containing device is configured to be placed in the fluidic chamber and replaceable with another urease-containing device for hydrolysis of urea in the fluidic chamber as the urea-containing liquid flows through the fluidic chamber; and
further such that the reference electrode, the cathode and the anode contact the urea-containing liquid in the fluidic chamber for measuring electric current caused by hydrolysis of urea in the fluidic chamber.

2. The urea sensor of claim 1, wherein the fluidic chamber is made of a synthetic resin material.

3. The urea sensor of claim 2, wherein the fluidic chamber is made of PDMS (polydimethylsiloxane).

4. The urea sensor of claim 1, wherein the urease-containing device composes urease and an insoluble porous support to which the urease is immobilized, wherein the insoluble porous support is made of at least one biocompatible material selected from the group consisting of fucoidan, collagen, alginate, chitosan, hyaluronic acid, silk fibroin, polyimide, polyamic acid, polycaprolactone, polyetherimide, nylon, polyaramid, polyvinyl alcohol, polyvinylpyrrolidone, poly-benzyl-glutamate, polyphenylene terephthalamide, polyaniline, polyacrylonitrile, polyethylene oxide, polystyrene, cellulose, polyacrylate, polymethyl methacrylate, polylactic acid (PLA), polyglycolic acid (PGA), polylactic acid-co-polyglycolic acid (PLGA), poly{polyethylene oxide)terephthalate-co-butylene terephthalate} (PEOT/PBT), polyphosphoester (PPE), polyphosphazene (PPA), polyanhydride (PA), poly(ortho ester) (POE), poly(propylene fumarate)-diacrylate (PPF-DA), and poly(ethylene glycol) diacrylate (PEG-DA).

5. The urea sensor of claim 1, wherein the urease-containing device comprises urease and an insoluble porous support to which the urease is immobilized, wherein the insoluble porous support is made of porous silk fibrobin.

6. The urea sensor module of claim 1, wherein the reference electrode, the cathode, and the anode are screen-printed on a surface of the electrode strip, wherein the reference electrode, the cathode, and the anode are configured to be connected to a potentiostat for a cyclic voltammetric analysis.

7. The urea sensor of claim 1, wherein the urease-containing device is in the form of a film.

8. (canceled)

9. The urea sensor of claim 1, wherein the urease-containing device and the fluidic chamber are configured to have generally the same cross-section such that the urease-containing device is stable inside the fluidic chamber.

10. The urea sensor of claim 1, wherein the fluidic chamber comprises a cylindrical space, wherein the device is in the form of a disc that can be placed inside the cylindrical space of the fluidic chamber.

11. A method of detecting urea in urea-containing liquid, the method comprising:
providing the urea sensor of claim 1;
supplying urea-containing liquid to the liquid inflow tube to flow the urea-containing liquid through the fluidic chamber and to get the urea-containing liquid discharged via the liquid outflow tube;
detecting a concentration of urea of the urea-containing liquid flowing through the fluidic chamber while the urease-containing device is kept in the fluidic chamber;
subsequently, replacing the urease-containing device with another urease-containing device; and
subsequently, detecting a concentration of urea of the urea-containing liquid flowing through the fluidic chamber while the other urease-containing device is kept in the fluidic chamber.

12. The method of claim 11, wherein the fluidic chamber is made of PDMS (polydimethylsiloxane).

13. The method of claim 11, wherein the urease-containing device comprises urease and an insoluble porous support to which the urease is immobilized, wherein the insoluble porous support is made of at least one biocompatible material selected from the group consisting of fucoidan, collagen, alginate, chitosan, hyaluronic acid, silk fibroin, polyimide, polyamic acid, polycaprolactone, polyetherimide, nylon, polyaramid, polyvinyl alcohol, polyvinylpyrrolidone, poly-benzyl-glutamate, polyphenylene terephthalamide, polyaniline, polyacrylonitrile, polyethylene oxide, polystyrene, cellulose, polyacrylate, polymethyl methacrylate, polylactic acid (PLA), polyglycolic acid (PGA), polylactic acid-co-polyglycolic acid (PLGA), poly {poly(ethylene oxide)terephthalate-co-butylene terephthalate} (PEOT/PBT), polyphosphoester (PPE), polyphosphazene (PPA), polyanhydride (PA), poly(ortho ester) (POE), poly(propylene fumarate)-diacrylate (PPF-DA), and poly(ethylene glycol) diacrylate (PEG-DA).

14. The method of claim 11, wherein the urease-containing device comprises urease and an insoluble porous support to which the urease is immobilized, wherein the insoluble porous support is made of porous silk fibrobin.

15. The method of claim 11, wherein the reference electrode, the cathode, and the anode are screen-printed on a surface of the electrode strip, wherein the reference electrode, the cathode, and the anode are configured to be connected to a potentiostat for a cyclic voltammetric analysis.

16. The method of claim 11, wherein the urease-containing device is in the form of a film.

17. The method of claim 11, wherein the urease-containing device and the fluidic chamber are configured to have generally the same cross-section such that the urease-containing are stable inside the fluidic chamber.

18. The method of claim 11, wherein the fluidic chamber comprises a cylindrical space, wherein the device is in the form of a disc that can be placed inside the cylindrical space of the fluidic chamber.
